(19) United States
(12) Patent Application Publication
(10) Pub. No.: US 2017/0340390 A1
(43) Pub. Date: Nov. 30, 2017

(54) Computer-Assisted Osteocutaneous Free Flap Reconstruction

(71) Applicant: University of Washington, Seattle, WA (US)

(72) Inventors: Richard A. Harbison, Seattle, WA (US); Yangming Li, Seattle, WA (US); Randall Bly, Seattle, WA (US); Nava Aghdasi, Seattle, WA (US); Blake Hannaford, Seattle, WA (US); Kristen S. Moe, Seattle, WA (US); Jeffrey J. Houlton, Seattle, WA (US)

(21) Appl. No.: 15/591,463

(22) Filed: May 10, 2017

Related U.S. Application Data
(60) Provisional application No. 62/342,318, filed on May 27, 2016.

(51) Int. Cl. A61B 34/10; G06T 7/33; G06T 19/20

(52) U.S. Cl. CPC A61B 34/10; G06T 19/20; G06T 7/337; G06T 2207/20221; A61B 2034/104; A61B 2034/105; A61B 2034/107; G06T 2207/30008; G06T 2219/2004

(57) ABSTRACT

Example methods and systems to facilitate osteocutaneous free flap reconstructions are provided. One example method involves causing a surgical navigation system to display a representation of a patient undergoing surgery; receiving input data representing one or more osteotomies made during the surgery to form a defect within the patient; determining a donor site for a bone graft having a contour that corresponds to the defect within the patient using a geometric alignment algorithm to identify, as the donor site, a portion of the bone graft that virtually aligns with the defect; generating a virtual template representing the bone graft; displaying the generated virtual template representing the bone graft within the representation of the patient as a navigational guide for harvesting of the bone graft; and displaying the generated virtual template positioned into the defect within the representation as a navigational guide for reconstructing the defect using the harvested bone graft.

### RELATED APPLICATIONS

This application claims priority to U.S. Provisional Patent Application Ser. No. 62/342,318 filed May 27, 2016 entitled Computer Assisted Maxillary Reconstruction, which is incorporated herein in its entirety.

### BACKGROUND

Unless otherwise indicated herein, the materials described in this section are not prior art to the claims in this application and are not admitted to be prior art by inclusion in this section.

Osteocutaneous free flap reconstruction refers to the use of bone-containing tissue from one part of a patient's body to repair a defect in another part of their body. Osteocutaneous free flaps, such the scapula, iliac crest, and fibula free flaps, are used in the reconstruction of oncologic, congenital, and traumatic defects, among others. Reconstruction of such defects often has profound functional and aesthetic implications for patients and their families.

For instance, reconstruction of a maxillectomy defect has the potential to restore important functions such as supporting the orbit (i.e., eye) and dentition (i.e., teeth), separating the oral and nasal cavities, and providing a framework for facial musculature/contour. Maxillectomy defects may result from a maxillectomy to remove tumor-involved bone. Tumors requiring maxillectomy often extend beyond the bony walls of the maxilla necessitating resection of adjacent tissues including the palate, midface, and orbit, thereby creating a total maxillectomy defect. Orbit-preserving total maxillectomy defects (known as Brown type 3) are considered by many to be among the most difficult reconstructive challenges of the head and neck. If insufficient orbital support is provided along the orbital floor or rim, a variety of ocular complications can result.

At least some of the difficulty in reconstructing oncologic, congenital, and traumatic defects can be attributed to the variability involved at all stages of the procedure. Variation in the contours of a defect of any given type can be expected, given the natural variation among patients and the conditions that lead to such defects. Further, when planning and performing a reconstruction, surgeons often have latitude in both selecting a suitable donor site and positioning of the osteocutaneous free flap within the defect.

Given this difficulty, several technological advances have been developed to enhance osteocutaneous free flap reconstruction including computer-aided planning and surgical navigation. For instance, some computer-aided planning techniques involve using stereolithographic models (i.e., 3-D printed models) in advance of a surgical procedure to help plan osteotomies, aid plate shaping, and/or facilitate intraoperative bone graft contouring. However, use of computer-aided planning is limited by the inability to adjust the operative plan in “real-time” during the surgical procedure. Alternatively, surgical navigation can be used for “real-time” reconstructive planning but current techniques still involve a surgeon determining the optimal positioning of a reconstructive plate or bone based on visual confirmation. As such, outcomes are primarily dependent on the surgeon's skill and experience.

### SUMMARY

Described herein are methods and systems for computer-assisted osteocutaneous free flap reconstruction. The methods and systems may be employed during surgical procedures to facilitate reconstruction of various defects, such as oncologic, congenital, and traumatic defects, using osteocutaneous free flaps. For instance, example methods and systems may be used intraoperatively to reconstruct maxillectomy defects resulting from tumor resection, among other examples.

Example implementations may involve computer-assisted selection of a donor site for a bone graft using virtual representations of the patient and one or more osteotomies made during the surgery to form a defect within the patient. During surgery, a computing system (e.g., a surgical navigation system) may cause a graphical display to display a three-dimensional representation of a patient undergoing surgery using imaging data (e.g., CT or MRI imaging data) registered with the computing system. Based on this three-dimensional representation, a surgeon may provide input to the computing system (e.g., using a navigation pointer on the displayed three-dimensional representation) to designate the actual osteotomies performed during surgery. By using a virtual representation of the actual osteotomies performed during surgery, computer-assisted selection of a donor site can be based on the actual defect within the patient, rather than on a pre-operatively planned defect (which might differ from the actual defect).

Using a geometric alignment algorithm, the computing system may determine a donor site for the bone graft using the virtual representations of the patient and the one or more osteotomies made during the surgery as input to the algorithm. Example geometric alignment algorithms contemplated herein produce an alignment between two geometric shapes. One geometric shape used as input to such an algorithm may be a donor bone (e.g., a portion of the virtual representation of a patient corresponding to a donor bone). Another geometric shape used as input to the algorithm may be a representation of the patient following one or more osteotomies made during the surgery (so as to represent the actual defect formed by the osteotomies). Given such input, the output of the geometric alignment algorithm may produce a portion of the donor bone suitable for use as a donor site for the bone graft.

One example geometric alignment algorithm is the iterative closest point algorithm, which is designed to minimize the distance between a reference point cloud (representing a first geometric shape) and a source point cloud (representing a second geometric shape). In an example, a reference point cloud (representing the actual defect within the patient) and a source point cloud (representing a possible donor bone) are provided as input to an implementation of an iterative closest point algorithm. Given this input, the algorithm repeatedly transforms the source point cloud (e.g., the donor bone) to identify a portion of the donor bone conforming to the contour of the defect within the patient, which then may be selected as the donor site. Other types of geometric alignment algorithms may be used to determine geometric alignment between the defect and the donor bone as well.

Having determined a donor site for the bone graft, the computing system may generate a virtual template representing the bone graft. This virtual template, or model, can be displayed within the three-dimensional representation of the patient as a navigational guide for harvesting of the bone graft. For instance, a surgical navigation system can display the virtual template as a marked portion of the donor bone, allowing a surgeon to resect the bone graft from the donor bone using the marked portion as a guide. Such guidance may promote a match between the actual bone graft and the determined donor site.

The virtual template may also aid in positioning the bone graft within the defect. For instance, the computing system may cause the graphical display to display the generated virtual template positioned into the defect within the three-dimensional representation of the patient. The positioning of the bone graft relative to the defect is previously determined using the geometric alignment. Given this known positioning, the computing system can orient the bone graft within the defect, thereby providing the surgeon a navigational guide for reconstructing the defect using the harvested bone graft. Since the virtual template is used as a guide in resecting the bone graft, the resected bone graft is likely to correspond closely to the virtual template, thereby improving the usefulness of the virtual template as a guide in positioning the bone graft.

In one aspect, a method is provided. The method may involve: causing a graphical display of a surgical navigation system to display imaging data comprising a three-dimensional representation of a patient undergoing surgery; receiving input data representing one or more osteotomies made during the surgery to form a defect within the patient; determining, via one or more processors, a donor site for a bone graft having a contour that corresponds to the defect within the patient, wherein determining the donor site for the bone graft having the contour that corresponds to the defect within the patient comprises using a geometric alignment algorithm to identify, as the donor site, a portion of the bone graft that virtually aligns with the defect; generating a virtual template representing the bone graft; causing the graphical display of the surgical navigation system to display the generated virtual template representing the bone graft within the three-dimensional representation of the patient as a navigational guide for harvesting of the bone graft; and causing the graphical display of the surgical navigation system to display the generated virtual template positioned into the defect within the three-dimensional representation of the patient as a navigational guide for reconstructing the defect using the harvested bone graft.

In another aspect, a system is provided. The system may include: one or more processors; a communications interface to a surgical navigation system; and a tangible non-transitory computer-readable medium having computer-executable instructions stored thereon that are executable by one or more processors of a computing device to cause the computing device to perform functions. The functions may include causing a graphical display of a surgical navigation system to display imaging data comprising a three-dimensional representation of a patient undergoing surgery; receiving input data representing one or more osteotomies made during the surgery to form a defect within the patient; determining, via one or more processors, a donor site for a bone graft having a contour that corresponds to the defect within the patient, wherein determining the donor site for the bone graft having the contour that corresponds to the defect within the patient comprises using a geometric alignment algorithm to identify, as the donor site, a portion of the bone graft that virtually aligns with the defect; generating a virtual template representing the bone graft; causing the graphical display of the surgical navigation system to display the generated virtual template representing the bone graft within the three-dimensional representation of the patient as a navigational guide for harvesting of the bone graft; and causing the graphical display of the surgical navigation system to display the generated virtual template positioned into the defect within the three-dimensional representation of the patient as a navigational guide for reconstructing the defect using the harvested bone graft.

In another aspect, a physical, non-transitory computer-readable medium is provided. The physical computer-readable medium may include instructions that are executable by a computing system to cause the computing system to perform functions. The functions include: causing a graphical display of a surgical navigation system to display imaging data comprising a three-dimensional representation of a patient undergoing surgery; receiving input data representing one or more osteotomies made during the surgery to form a defect within the patient; determining, via one or more processors, a donor site for a bone graft having a contour that corresponds to the defect within the patient, wherein determining the donor site for the bone graft having the contour that corresponds to the defect within the patient comprises using a geometric alignment algorithm to identify, as the donor site, a portion of the bone graft that virtually aligns with the defect; generating a virtual template representing the bone graft; causing the graphical display of the surgical navigation system to display the generated virtual template representing the bone graft within the three-dimensional representation of the patient as a navigational guide for harvesting of the bone graft; and causing the graphical display of the surgical navigation system to display the generated virtual template positioned into the defect within the three-dimensional representation of the patient as a navigational guide for reconstructing the defect using the harvested bone graft.

These as well as other aspects, advantages, and alternatives, will become apparent to those of ordinary skill in the art by reading the following detailed description, with reference where appropriate to the accompanying drawings.

### BRIEF DESCRIPTION OF THE FIGURES

FIG. 1 shows a simplified block diagram of a computing system, in accordance with an example embodiment.

FIG. 2 shows an example three-dimensional representation of a patient.

FIG. 3A shows an example virtual representation of a patient.

FIG. 3B shows another example virtual representation of a patient.

FIG. 4 shows an illustrative virtual representation of a donor bone and defect using point clouds.

FIG. 5A shows an illustrative virtual representation of a donor bone and defect using point clouds.

FIG. 5B shows an illustrative virtual representation of the donor bone, defect, and a donor site using point clouds.

FIG. 6 shows another illustrative virtual representation of a donor bone and defect using point clouds.

FIG. 7 shows a further illustrative virtual representation of a donor bone and defect using point clouds.

FIG. 8 shows an example stereolithographic model.

FIG. 9 shows an example user interface to facilitate osteocutaneous free flap reconstruction.

FIG. 10 shows an example user interface to facilitate osteocutaneous free flap reconstruction.

FIG. 11 shows an example user interface to facilitate osteocutaneous free flap reconstruction.

FIG. 12 shows an illustrative method to facilitate osteocutaneous free flap reconstruction.

### DETAILED DESCRIPTION

In the following detailed description, reference is made to the accompanying figures, which form a part thereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the spirit or scope of the subject matter presented herein. It will be readily understood that aspects of the present disclosure, as generally described herein, and illustrated in the figures, can be arranged, substituted, combined, separated, and/or designed in a wide variety of different configurations, all of which are explicitly contemplated herein.

### 1. EXAMPLE ARCHITECTURE

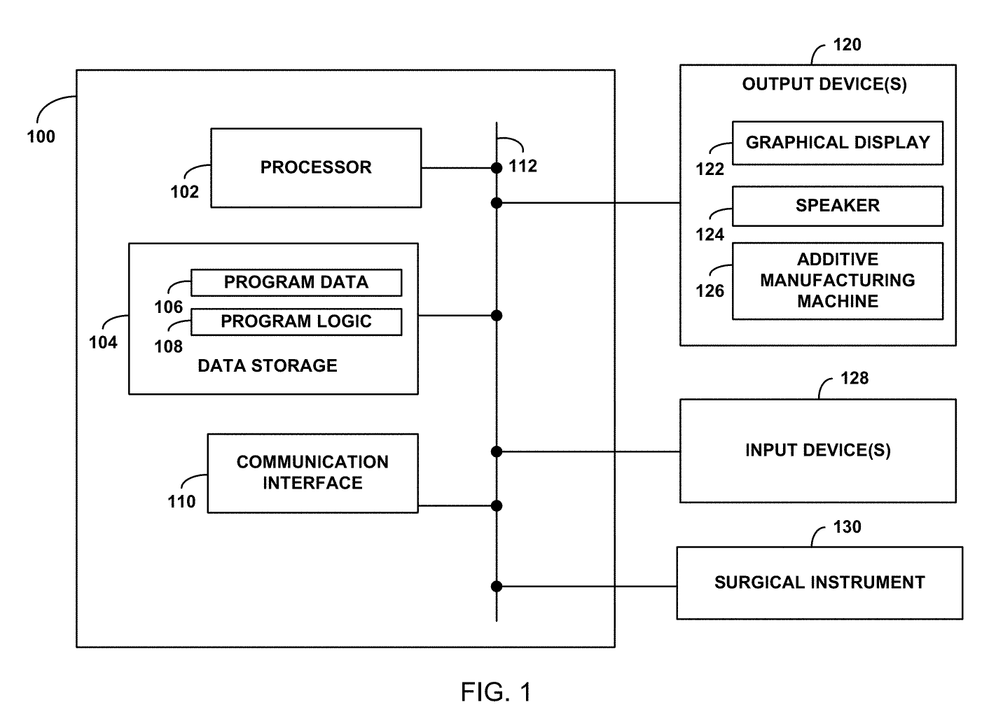

FIG. 1 shows a simplified block diagram of an example computing system 100 in which the example techniques can be implemented. It should be understood that this and other arrangements described herein are set forth only as examples. Those skilled in the art will appreciate that other arrangements and elements (e.g., machines, interfaces, functions, orders, and groupings of functions, etc.) can be used instead and that some elements may be omitted altogether. Further, many of the elements described herein are functional entities that may be implemented as discrete or distributed components or in conjunction with other components, and in any suitable combination and location. Various functions described herein as being performed by one or more entities may be carried out by hardware, firmware, and/or software. And various functions described herein may be carried out by a processor executing instructions stored in memory.

As shown in FIG. 1, computing system 100 may include processor 102, data storage 104, and communication interface 110, all linked together via system bus, network, or other connection mechanism 112.

Processor 102 may include one or more general purpose microprocessors and/or one or more dedicated signal processors and may be integrated in whole or in part with communication interface 110. Data storage 104 may include tangible, physical memory and/or other storage components, such as optical, magnetic, organic or other memory disc storage, which can be volatile and/or non-volatile, internal and/or external, and integrated in whole or in part with processor 102. Data storage 104 may be arranged to contain (i) program data 106 and (ii) program logic 108. Although these components are described herein as separate data storage elements, the elements could just as well be physically integrated together or distributed in various other ways. For example, program data 106 may be maintained in data storage 104 separate from program logic 108, for easy updating and reference by program logic 108.

Communication interface 110 typically functions to communicatively couple computing system 100 to networks. As such, communication interface 110 may include a wired (e.g., Ethernet) and/or wireless (e.g., Wi-Fi) packet-data interface, for communicating with other devices, entities, and/or networks. Computing system 100 may also include multiple interfaces 110, such as one through which computing system 100 sends communication, and one through which computing system 100 receives communication.

Computing system 100 may also include, or may be otherwise communicatively coupled to, output device(s) 120. Output device(s) 120 may include one or more elements for providing output, for example, one or more graphical displays 122, a speaker 124, and/or an additive manufacturing machine 126 (also known as a three-dimensional printer or a stereolithography machine). In operation, output device 120 may be configured to display a graphical user interface (GUI) via graphical display 122, corresponding to use of such a GUI.

Computing system 100 may further include, or may be otherwise communicatively coupled to, input device(s) 128. Input device(s) 128 may include one or more elements for receiving input, such as a keyboard and mouse. In some embodiments, input device 128 may include a touch-sensitive display, which may be incorporated into graphical display 122. The touch-sensitive display may support capacitive or resistive sensing using a finger or pen for example.

Computing system 100 may further be communicatively coupled to a surgical instrument(s) 130. The surgical instrument(s) 130 may include any type(s) of surgical instrument, such as an instrument for resection and/or an instrument for delivering therapeutics such as in chemotherapy or radiation therapy, among many other examples. The surgical instrument 130 may include a system that provides tracking of the position of the surgical instrument 130.

Computing system 100 may be part of a surgical navigation system or communicatively coupled to a surgical navigation system. Example surgical navigation systems include a computing system (possibly computing system 100, or possibly another computing system coupled to computing system 100) connected to a graphical display (e.g., graphical display 122). Example surgical navigation systems may also include an input device, such as a navigation pointer (e.g., input device(s) 126). Some surgical navigation systems include one or more surgical instruments that are tracked by the surgical navigation system (e.g., surgical instrument(s) 130). Example surgical navigation systems include software to facilitate surgical operations, such as by displaying on the graphical display a representation of a patient undergoing surgery, perhaps based on radiographic imaging data.

Commercially-available surgical navigation systems include the STEALTHSTATION from MEDTRONIC® and the STRYKER NAVIGATION PLATFORM from STRYKER®, among many other examples.

This disclosure describes computing system 100 as performing various methods and functions to carry out example implementations described herein. In some examples, computing system 100 may perform these functions and/or methods in response to user input directing computing system 100 to perform the functions and/or methods (e.g., user input via input device(s) 128). However, the claims should not be interpreted to require user input, unless explicitly recited.

As noted above, in some embodiments, the disclosed techniques may be implemented by computer program instructions encoded on a physical, and/or non-transitory, computer-readable storage media in a machine-readable format, or on other non-transitory media or articles of manufacture. For instance, the computer program instructions may be encoded on a hard disk drive, a Compact Disc (CD), a Digital Video Disk (DVD), a digital tape, memory, etc. In some implementations, the signal bearing medium 202 may encompass a computer-recordable medium 208, such as, but not limited to, memory, read/write (R/W) CDs, R/W DVDs, etc.

The computer program instructions may be, for example, computer executable and/or logic implemented instructions. In some examples, a computing system such as the computing system 100 of FIG. 1 may be configured to provide various operations, functions, or actions in response to the computer program instructions conveyed to the computing system 100 by one or more of a computer readable medium, a computer recordable medium, and/or a communications medium.

### 2. EXAMPLE IMPLEMENTATIONS

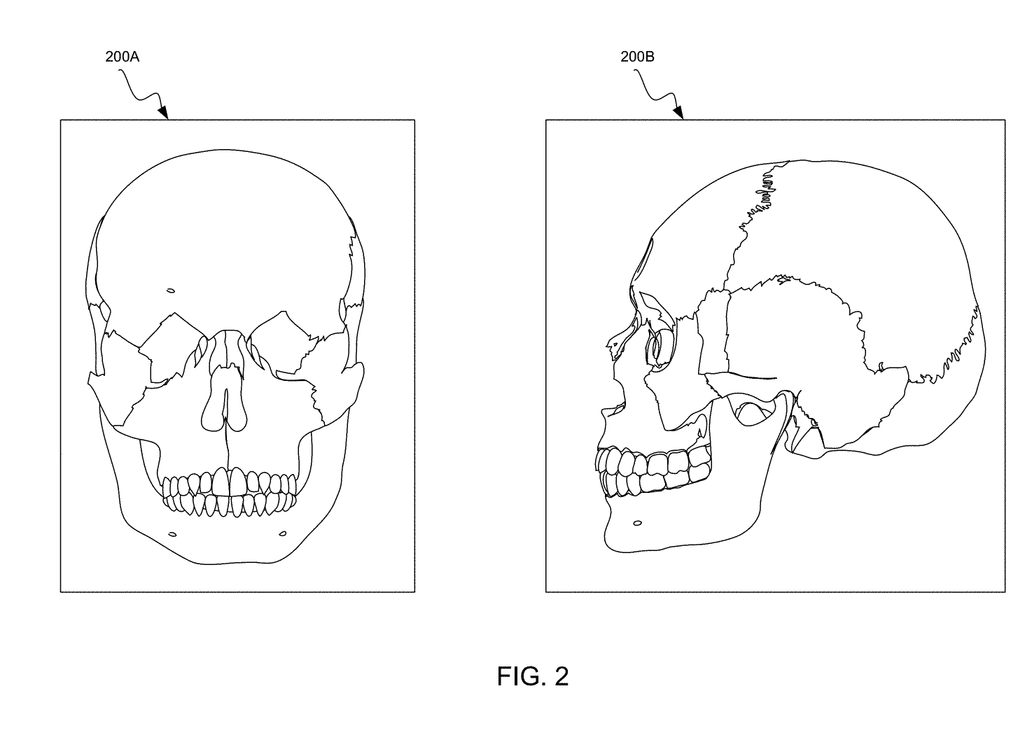

FIG. 2 illustrates a three-dimensional representation (i.e., a virtual image or model) of a patient (i.e., a patient's skull). In particular, FIG. 2 includes an anterior view 200A and a lateral view 200B of the three-dimensional representation. A surgical navigation system may generate such a three-dimensional representation of a patient using imaging data from a medical imaging machine, such as a magnetic resonance imaging (MRI) machine, a positron emission tomography (PET) machine, a computed tomography (CT) machine, and/or an X-ray machine. In practice, patients often undergo pre-operative imaging. For instance, patients may undergo baseline CT scanning as part of an oncologic workup.

Computing system 100 in FIG. 1 may receive such imaging data over the system bus, network, or other connection mechanism 112. In some embodiments, the computing system may receive the imaging data from another computing system via a network over communication interface 110. For instance, the computing system may receive the imaging data from a Digital Imaging and Communications in Medicine (DICOM) storage system.

Alternatively, the computing system may receive the imaging data via a transfer from a data storage device, such as a hard disk drive or a USB flash drive. In other embodiments, the computing system may receive the imaging data via a transfer from a data storage medium, such as a CD-ROM disk. Many other examples are possible as well.

As noted above, the representation may be generated from imaging data. The imaging data may include one or more images produced using one or more of a variety of medical imaging techniques such as Mill, PET, or CT. The imaging data may include images from different perspectives, such as sagittal, coronal, or transverse.

In some cases, the representation may be a three dimensional (3-D) representation. A 3-D representation may be provided from a set of medical images, known as a scan. For example, the computing system may combine multiple two-dimensional (2-D) images as layers to form a three-dimensional representation. In other embodiments, the medical imaging machine may produce a three-dimensional representation.

In some embodiments, the imaging data indicates signal intensity. Signal intensity may vary based on the density of the imaged subject matter. Different anatomical features within the representation may have different signal intensities, which appear in contrast (e.g., lighter or darker) on the image, thereby distinguishing the anatomical features. An image may have a pixel resolution, such as 512 pixels by 512 pixels, for a two-dimensional image. Or, where the image is 3-D, the image may have a voxel resolution, such as 512 voxels by 512 voxels by 66 voxels.

A pixel may represent a physical region within the image. For example, a pixel may represent a physical region of 0.8×0.8 mm. Therefore, the pixel is an approximation of that physical region. Likewise, a voxel may define a physical volume; for example, a volume of 0.8×0.8×7 mm. Because each pixel is an approximation of a physical region, each pixel may have a physical location. Such a physical location may be represented by a 2-D or 3-D coordinate system.

Each pixel in an image may have a signal intensity sample (signal intensity) associated with that respective pixel. The signal intensity associated with that respective pixel represents the amplitude of the signal at one point. However, it should be understood that a pixel is an approximation of a region. Therefore, the imaging data may be a 2-D or 3-D array of signal intensity data. Such an array may be referred to as a point cloud or image matrix.

When generating the three-dimensional representation, the computing system may define a coordinate system with respect to the representation. For instance, if the representation includes one or more two-dimensional (2-D) images, the computing system may define a 2-D coordinate system. Alternatively, the computing system may define a 3-D coordinate system. The origin of the coordinate system may be any point within the representation.

In some implementations, individual anatomical features of the patient may be defined within the three-dimensional representation. For instance, a patient's skeletal system, and/or individual bones thereof, have a distinct signal intensity compared with the surrounding issue. As such, certain portions of the three-dimensional representation may be defined as their corresponding features. For instance, these features may be defined as objects with a user interface of a surgical navigation system.

The surgical navigation system may cause the graphical display (e.g., graphical display 122) to present a user interface including the three-dimensional representation. During surgery, the patient may be registered with the surgical navigation system so that the three-dimensional representation of the patient and any virtual representations of surgical instruments tracked by the surgical navigation system (e.g., surgical instrument(s) 130) are calibrated to the same coordinate system as the actual patient and surgical instruments. In some implementations, light-emitting diodes are positioned on the patient to provide tracking markers for an optical imaging device of the surgical navigation system.

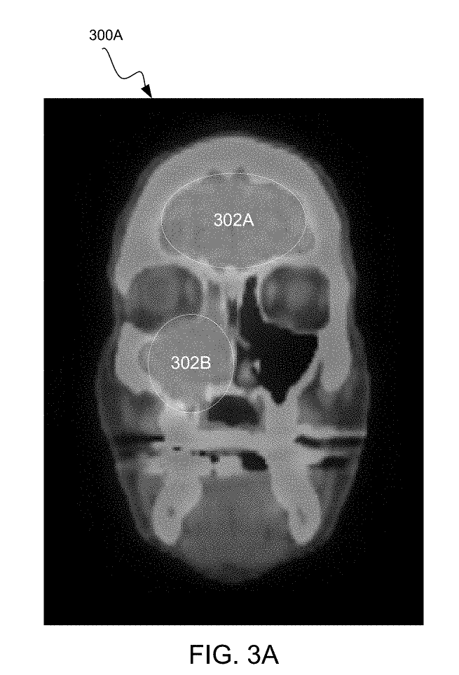

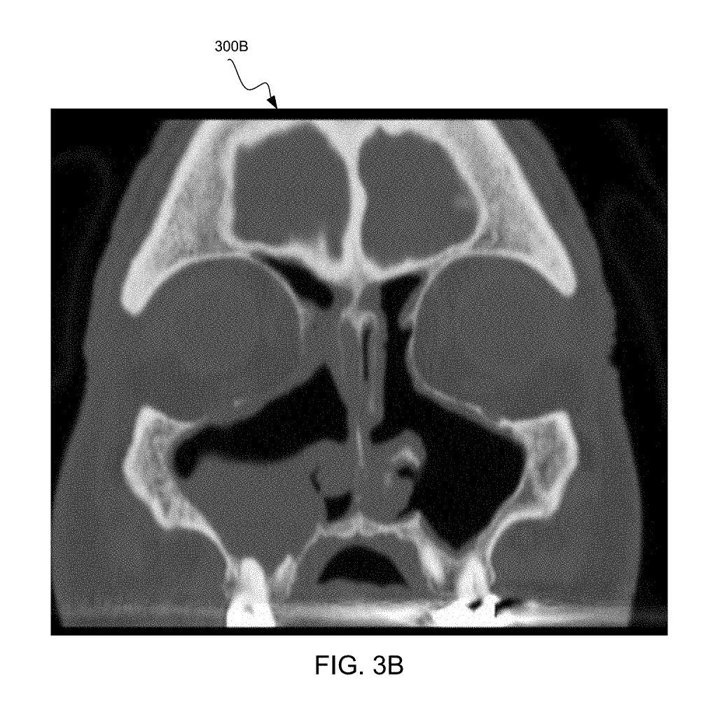

FIGS. 3A and 3B illustrate example representations 300A and 300B, respectively, as may be presented on graphical display 122 by computing system 100. Example representations 300A and 300B may be generated from imaging data, such as the imaging data described above. Although the representation of a patient may be three-dimensional, when displayed on a graphical display, the display may show a two-dimensional view (i.e., a “slice”) of the three-dimensional representation. Representations 300A and 300B depict two-dimensional “slices” of a patient's head.

A surgical navigation system may display such a representation of a patient when the patient is undergoing surgery, perhaps as a navigational aid to the surgeon performing the surgery. Referring back to FIG. 3A, representation 300A includes portions 300A and 300B, approximately marking tumor present in the patient's head. Displaying representations 300A and/or 300B using a surgical navigation system assists a surgeon in resecting this tumor.

Some tumors, such as the tumor shown in FIG. 3A, involve bone. To resect such a tumor, a surgeon may perform one or more osteotomies, thereby forming a defect in the patient. For instance, in resecting the tumor shown in FIG. 3A, the surgeon may perform a maxillectomy. Tumors requiring maxillectomy often extend beyond the bony walls of the maxilla necessitating resection of adjacent tissues including the palate, midface, and orbit, thereby creating a total maxillectomy defect. While an oncologic defect is described herein by way of example, techniques described herein also apply to traumatic and congenital defects, among others.

To reconstruct such a defect, a surgeon may use an osteocutaneous free flap. An osteocutaneous free flap is a bone graft harvested from another portion of the patient. As noted above, some example techniques described herein involve computer-assisted selection of the donor site for the osteocutaneous free flap.

To facilitate selection of the donor site for the osteocutaneous free flap, a surgeon may provide input to computing system 100 to define the defect within the patient. For instance, following one or more one or more osteotomies made during the surgery to form a defect within the patient, the surgeon may provide input via input device(s) 128 to define the one or more osteotomies (and thereby define the defect). Some surgical navigation systems include a navigation pointer that the surgeon can use to mark certain portions of displayed three-dimensional representation of a patient (e.g., to define a defect).

When such input is provided via input device(s) 128, computing system 100 may receive input data that represents the defect. For instance, where the surgeon performed one or more osteotomies to form a defect, computing system 100 may receive input data representing the one or more osteotomies made during the surgery to form the defect within the patient. As another example, computing system 100 may receive input data defining one or more dimensions of a desired reconstruction bone. The computing system may receive input data representing other types of defects as well, such as traumatic or congenital defects. Alternatively, the computing system may determine the defect fully or partially computationally based on the three-dimensional representation. Other examples are possible as well.

In some examples, the received input data represents vertices that define a defect. For instance, a surgeon may use a navigation pointer of the surgical navigation system to trace the coordinates of the resected bone and/or tissue, thereby creating trace lines with vertices that define the defect. The surgeon may define the defect intraoperatively, which may promote a closer match (or more consistent match) between the input data indicating the defect and the actual defect formed within the patient as compared with pre-operatively defined defects. Defects defined pre-operatively are less likely to match the actual defect since the surgical plan might be altered during surgery based on oncologic or aesthetic considerations, among others.

As noted above, a surgeon may use a navigation pointer of the surgical navigation system to define the defect. Some surgical navigation systems include a touch-sensitive graphical interface, which may display the three-dimensional representation of the patient. In such cases, the surgeon may provide touch input on the displayed three-dimensional representation of the patient using a finger or touch-sensitive pen or stylus, which is received as input data by the computing system. For instance, computing system 100 may receive input data representing input via a navigation pointer (e.g., a stylus) of the surgical navigation system to define three-dimensional coordinates on the three-dimensional representation of the patient. Such coordinates may indicate a defect by defining osteotomies or other surgical actions made during the surgery to form the defect within the patient.

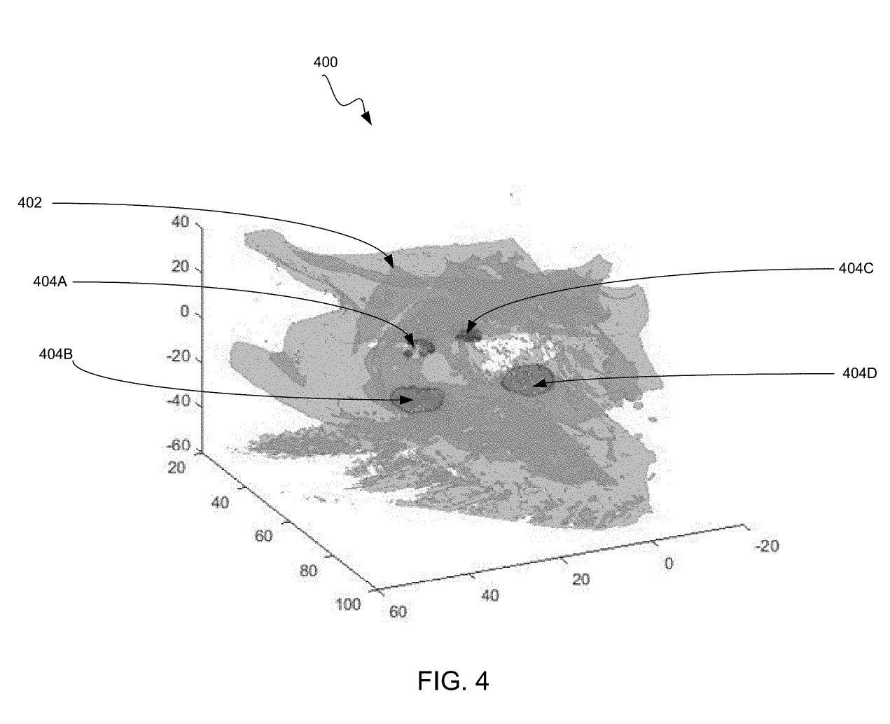

In some implementations, computing system 100 generates a first point cloud representing the defect within the patient. FIG. 4 shows a virtual representation 400, which depicts the patient's head and surgeon-defined vertices as respective point clouds. In particular, the point cloud 402 depicts the patient's maxillary bone and while point clouds 404A, 404B, 404C, and 404D depict respective defect vertices that were defined intraoperatively by a surgeon. Each of point clouds 404A, 404B, 404C, and 404D represent a respective osteotomy performed by the surgeon to form the defect (assuming that the vertices are accurately defined).

Computing system 100 may derive such point clouds from the three-dimensional representation of the patient and the input data defining the defect. As noted above, the imaging data may comprise one or more images, each of which may comprise pixels (or voxels) that represent respective physical regions within the image. Pixels corresponding to a particular type of tissue (e.g., bone) will have similar values (e.g., signal intensities that are within a given range). As such, the computing system 100 may generate point clouds using the respective values associated with each pixel, perhaps by generating point clouds that include pixels having associated values within a given range.

After the defect within the patient is indicated, computing system 100 may determine a donor site for a bone graft corresponding to the defect within the patient. An osteocutaneous free flap having a contour that corresponds to the defect within the patient may be considered suitable for reconstruction of the defect. As such, computing system 100 may seek to identify a portion of a donor bone having such a contour for the donor site.

To determine a donor site for a bone graft, computing system 100 may use a geometric alignment algorithm. Various geometric alignment algorithms accept geometric shapes as input to produce output indicative of an alignment between the two geometric shapes. Example techniques involve representing the defect and the donor bone digitally as respective geometric shapes. When provided as input to example geometric alignment algorithms, the algorithms produce output indicative of an alignment between the defect and the donor bone, thereby determining a portion of the donor bone having a contour suitable for reconstructing the defect.

In some implementations, the defect and the donor bone are represented as geometric shapes using respective point clouds. For instance, computing system 100 may generate a first point cloud representing the defect within the patient and a second point cloud representing a donor bone. To illustrate, referring back to FIG. 4, point clouds 404A, 404B, 404C, 404D represent a maxillary defect involving the orbital floor. Computing system 100 may generate a similar point cloud representing a donor bone from the imaging data representing the patient.

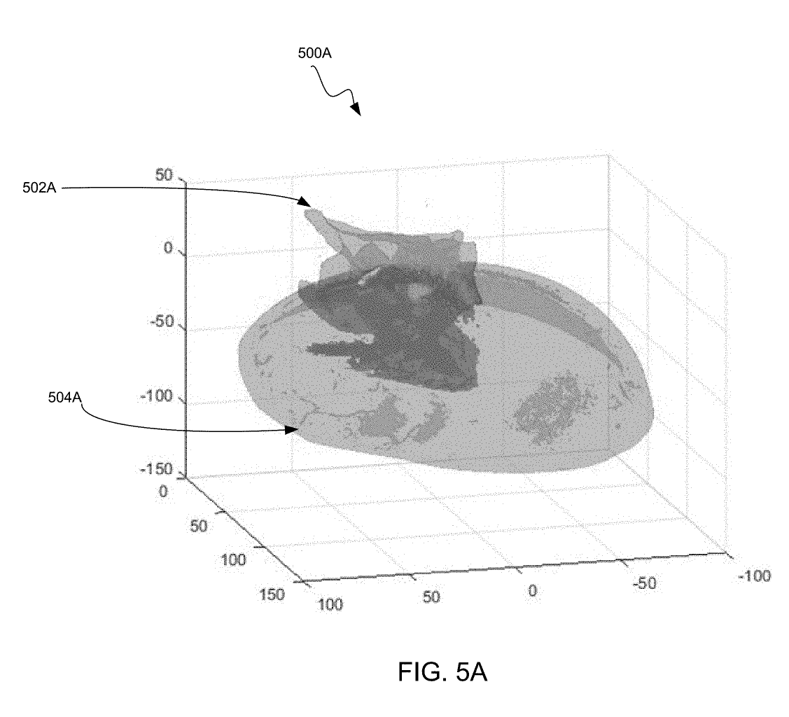

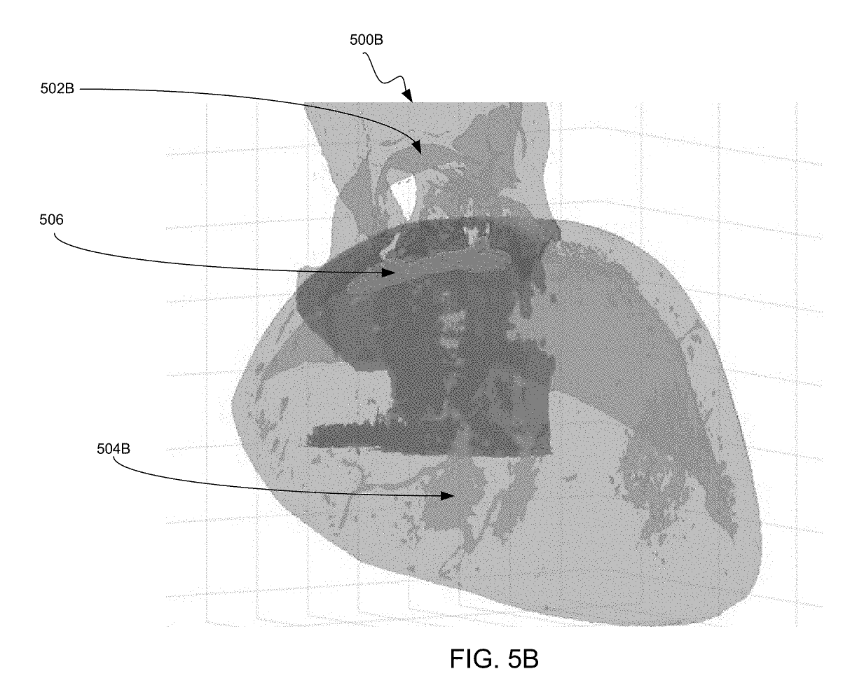

As another example, FIG. 5A shows a virtual representation 500A of defect and donor bones represented as respective point clouds. In particular, virtual representation 500A includes a point cloud 502A representing a right maxillary bone in which osteotomies were performed to form a defect within the patient. Virtual representation 500A also includes a point cloud 504A representing a portion of the patient's skull to be used as a donor bone.

Given a first point cloud representing the defect within the patient and a second point cloud representing a donor bone, computing system 100 may cause a geometric alignment algorithm to identify, as the donor site, a particular portion of the donor bone represented in the second point cloud that aligns with the defect represented in the first point cloud. In particular, computing system 100 may provide the first point cloud and second point cloud to the geometric alignment algorithm. From this input, the geometric alignment algorithm produces an output indicative of a geometric alignment between the first point cloud and second point cloud (i.e., between the defect and the donor bone). In particular, the output may indicate a portion of the second point cloud that geometrically aligns with the first point cloud. Given the degree of error allowed in the algorithm used, the indicated portion of the second point cloud may be assumed to have a contour that corresponds to the defect within the patient such that it is suitable for use in reconstructing the defect.

One example of a geometric alignment algorithm is the iterative closest point algorithm, which is an algorithm designed to minimize the difference between two clouds of points. In particular, the algorithm matches two point clouds (e.g., point clouds derived from radiographically imaging techniques) and minimizes the root mean square (RMS) conformance distances between the point clouds. As one example, an implementation of the iterative closest point algorithm was developed for MATLAB® by Wilm, which is available on the MATLAB® File Exchange. Other implementations are available as well.

Under iterative closest point algorithm, a first point cloud (referred to as the reference, or target), is kept fixed, while a second point cloud (referred to as the source) is transformed to match the reference. The algorithm iteratively revises the transformation (combination of translation and rotation) needed to minimize an error metric, usually the distance from the source to the reference point cloud. As such, the inputs to the algorithm are the reference and source point clouds, as well as criteria for stopping the iterations (e.g., a number of iterations to perform and/or a satisfactory degree of error). Given this input, example iterative closest point algorithms yield a geometric transformation that, when applied to the second point cloud, aligns the donor bone represented in the second point cloud with the defect represented in the first point cloud.

As such, in one example, when using the iterative closest point algorithm to determine a donor site from a target point cloud representing the defect within the patient and a source point cloud representing a donor bone, computing system 100 matches, for each point in the source point cloud, the closest point in the reference point cloud. Computing system 100 may estimate the combination of rotation and translation using a root mean square point-to-point distance metric minimization technique to align each source point to its match found in the previous matching step after weighting and rejecting outlier points. Computing system 100 then transform the source points using the obtained transformation. The computing system 100 may iterate these steps until the criteria for stopping the iterations are met. As another example, plane matching may be used as an alternative to point-to-point matching.

To illustrate, FIG. 5B shows a virtual representation 500B of defect and donor bones represented as respective point clouds. In particular, virtual representation 500B includes a point cloud 502B representing the right maxillary bone in which osteotomies were performed to form a defect within the patient. Virtual representation 500B also includes a point cloud 504B representing the portion of the patient's skull to be used as the donor bone. Given point cloud 502B and point cloud 504B as reference and source point cloud inputs to an iterative point algorithm, the iterative point algorithm yielded a donor site 506, which is a particular portion of point cloud 504B aligning with the defect in the right maxillary bone.

In addition to the donor site, the output of a geographic alignment algorithm such as the iterative closest point algorithm may indicate positioning and orientation of the free flap within the defect. As shown in FIG. 5B, point cloud 504B representing the portion of the patient's skull is transformed to orient the donor site 506 within the defect represented in the point cloud 502B. As such, the donor site 506 (i.e., the region of overlap between the donor bone and the defect) represents the portion of the donor bone for the surgeon to resect for free flap reconstruction as well as the positioning and orientation of the free flap within the defect.

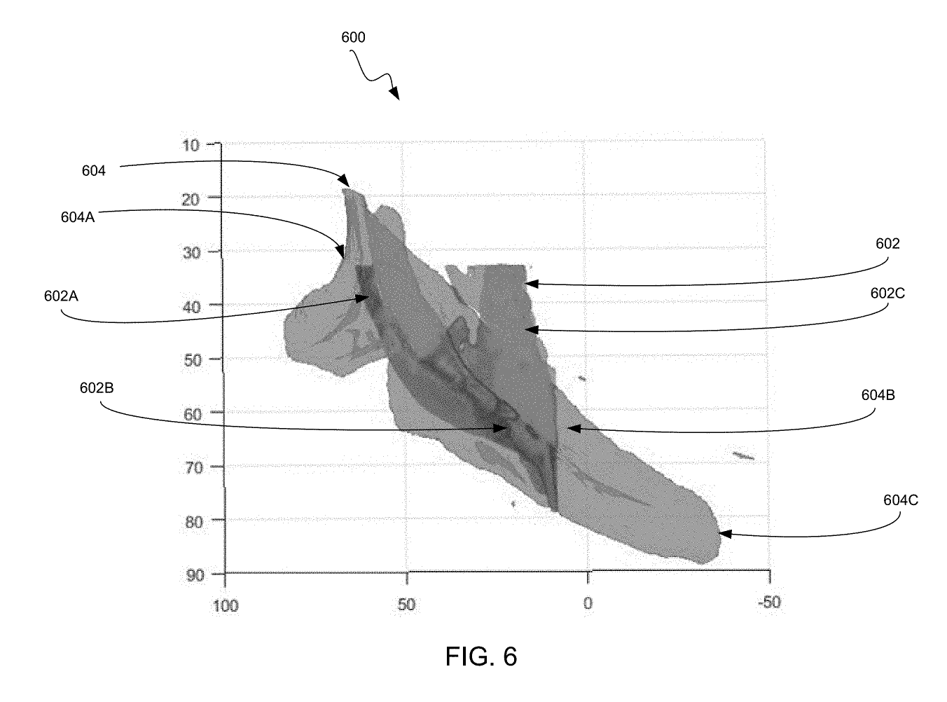

As another example, FIG. 6 shows a virtual representation 600 of a maxillary defect and donor bone represented as respective point clouds. In particular, virtual representation 600 includes a point cloud 602 representing the maxilla and a point cloud 604 representing a scapula. The view shown is looking down from top to bottom of the right maxillary bone. As shown, output from an iterative point algorithm suggested an alignment between the point cloud 602 and the point cloud 604 such the contour of the scapula matches with the maxillary defect. To illustrate the relative positioning, certain features of the maxilla and scapula are labeled, including the right maxilla 602A, anterior maxilla 602B, and posterior maxilla 602C and the shoulder joint 604A, midline 604B, and the scapula tip 604C.

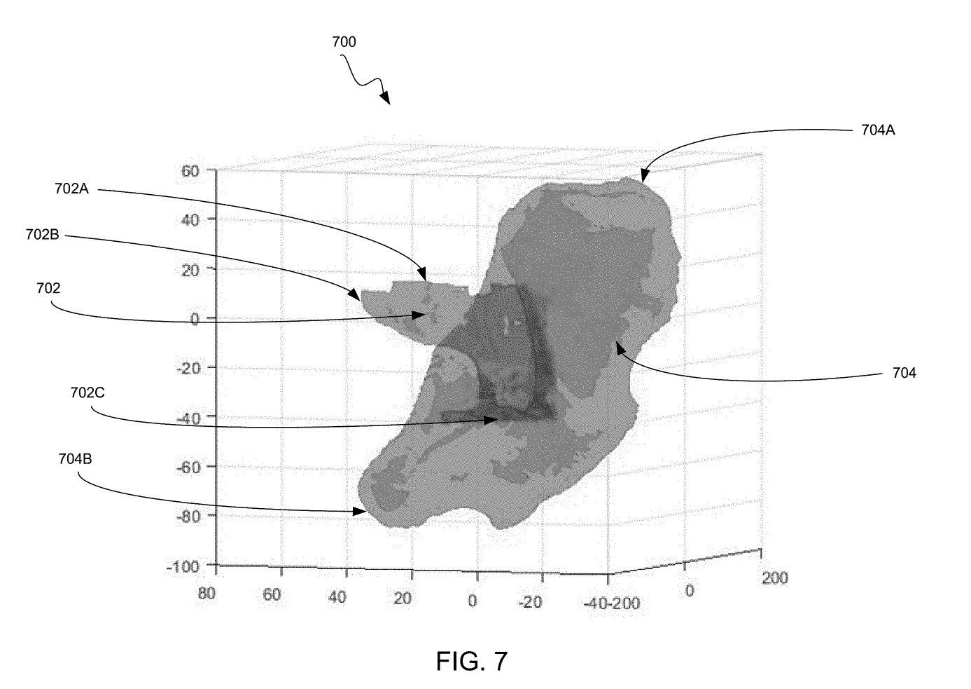

As a further example, FIG. 7 shows a virtual representation 700 of a maxillary defect in the right maxillary bone and donor bone represented as respective point clouds. In particular, virtual representation 700 includes a first point cloud 702 representing the maxilla and a second point cloud 704 representing a hip bone. As shown, output from an iterative point algorithm suggested an alignment between the point cloud 702 and point cloud 704 such the contour of the hip bone matches with the maxillary defect. To illustrate the relative positioning, certain features of the maxilla and hip bone are labeled, including the upper cheek 702A, right cheek 702B, and lower cheek 702C, as well as the upper hip bone 704A and lower hip bone 704B.

In some implementations, computing system 100 may receive input data indicating a donor bone for harvest. A surgeon may use input device(s) 128 to select a donor bone from the displayed three-dimensional representation. For instance, the surgeon may use a navigational pointer to select portions of the three-dimensional representation. As another example, one or more possible donor bones may be identified via one or more parameters defining coordinates of the three-dimensional representation. Other examples are possible as well.

Some iterations of the geometric alignment algorithm may output a donor site that is less desirable or not anatomically feasible for harvest. In such instances, the surgeon may re-select or otherwise modify the donor bone for harvest. For instance, if the geometric alignment algorithm suggests an inappropriate portion of skull as the donor site, the surgeon may update the parameters to define a portion of the skull as potential donor bone. The computing system may then cause the geometric alignment algorithm to repeat the donor site determination using this revised input.

In some instances, computing system 100 uses a geometric alignment algorithm on multiple donor bones, which may produce multiple prospective donor sites that virtually align with defect to varying degrees. In such cases, computing system 100 may generate multiple point clouds representing respective candidate donor bones of the patient. Then, computing system can repeat the algorithm using different inputs. For instance, with the iterative closest point algorithm, computing system 100 may iterate over respective source point clouds representing each donor bone while keeping the point cloud representing the defect as the reference point cloud.

After the prospective donor sites are identified, computer system 100 may select a portion of a given donor bone that virtually aligns with the defect as the donor site. In some cases, using the geometric alignment algorithm with multiple donor bones may assist computing system 100 in identifying a donor site that more closely matches the contour of the defect. For instance, computer system 100 can iterate over the multiple point clouds in an attempt to find a donor site having the least matching error among the candidate donor bones. As another example, computing system 100 can proceed to subsequent donor bones if no donor site with acceptable (e.g., below a threshold) error is found on a first donor bone. Various other criteria may be used to select the donor site as well.

In some instances, computing system 100 may apply a weighting function to the geometric alignment algorithms. This weighting function may cause the geometric alignment algorithm to increase a weight of a certain feature (or features) within the defect over other features when identifying a donor site. For instance, if the surgeon performed multiple osteotomies to resect a tumor and a number of bone features were exposed, a subset of these bone features may be weighted more heavily, if a close match between that subset of features and the free flap was desired for reconstruction. Various implementations of the iterative closest point algorithm have optional weighing functions. The weighing can be applied by defining a portion (or portions) of the point cloud representing the defect to weigh more heavily (as compared with other portions of the point cloud).

For instance, when determining a donor site for reconstructing a maxillectomy defect, certain maxillary features (e.g., the orbital rim and alveolar ridge) may be weighted more heavily, as orbital floor and rim support depend up on the reconstruction of these features. Lack of orbital floor and/or rim support following maxillary reconstruction may result in ocular complications including hypophthalmos (i.e., downward displacement of the eye), enophthalmos (i.e., posterior displacement of the eye), vertical dystopia (i.e., abnormal vertical positioning of the eyes), diplopia (i.e., double vision), and ectropion (i.e., outward turned eyelid). Therefore, determining a donor for a free flap that promotes reconstruction of these features may improve outcomes.

After the donor site is determined, computing system 100 generates a virtual template representing the bone graft, which may facilitate resection of the bone graft from the donor site and/or reconstruction of the defect using the bone graft, among other possible uses. The virtual template is a three-dimensional model of the bone graft. The computing system 100 may generate the virtual template based on the output of the geographic alignment algorithm.

For example, where an iterative point algorithm determines, as the donor site, an area of overlap between two point clouds (i.e., the defect and the donor bone), the portion of the donor bone within the area of overlap represents the portion of bone that will ultimately be used as the bone graft. A subset of the second point cloud representing this portion of the donor bone is extracted from the second point cloud to produce a point cloud representing the donor bone. Computing system 100 may generate a virtual template representing the bone graft from this point cloud, as the geometric shape of the point cloud corresponds to the bone graft. To illustrate, referring back to FIG. 5B, the donor site 506, which represents the area of overlap between the point clouds 502B and 504B, can be extracted to generate a virtual template representing a bone graft from the donor site 506.

In some implementations, computing system 100 may generate the virtual template by generating a stereolithographic model corresponding to the bone graft. The stereolithographic model may be generated from a point cloud or other three-dimensional representation of the bone graft. This stereolithographic model may be uploaded to the surgical navigation to function as a virtual template.

A stereolithographic model may facilitate use of the virtual template as a guide for resecting the donor bone. For instance, using the stereolithographic model of the bone graft as a guide, computing system 100 may generate lines to mark one or more osteotomies to resect the bone graft from the donor site.

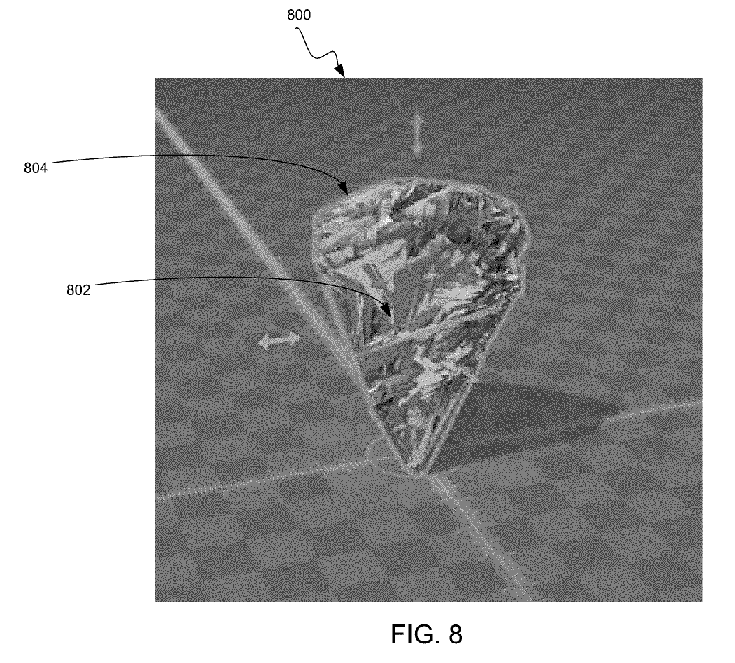

To illustrate, FIG. 8 includes a virtual representation 800 of a stereolithographic model 802, which is a virtual template representing a bone graft from a skull. Osteotomy lines 804 were generated around the stereolithographic model 802 using stereolithographic model 802 as a guide. These lines mark one or more osteotomies that a surgeon may perform to resect a bone graft corresponding to this stereolithographic model.

As a navigational guide for harvesting of the bone graft, computing system 100 may cause the graphical display of the surgical navigation system to display the generated virtual template representing the bone graft within the three-dimensional representation of the patient. For instance, computing system 100 may upload the generated virtual template to the surgical navigation system so as to display the generated virtual template concurrently with the three-dimensional representation of the patient undergoing surgery. Such display may facilitate resection of the bone graft, by providing the generated virtual template as a guide on the representation of the patient undergoing surgery.

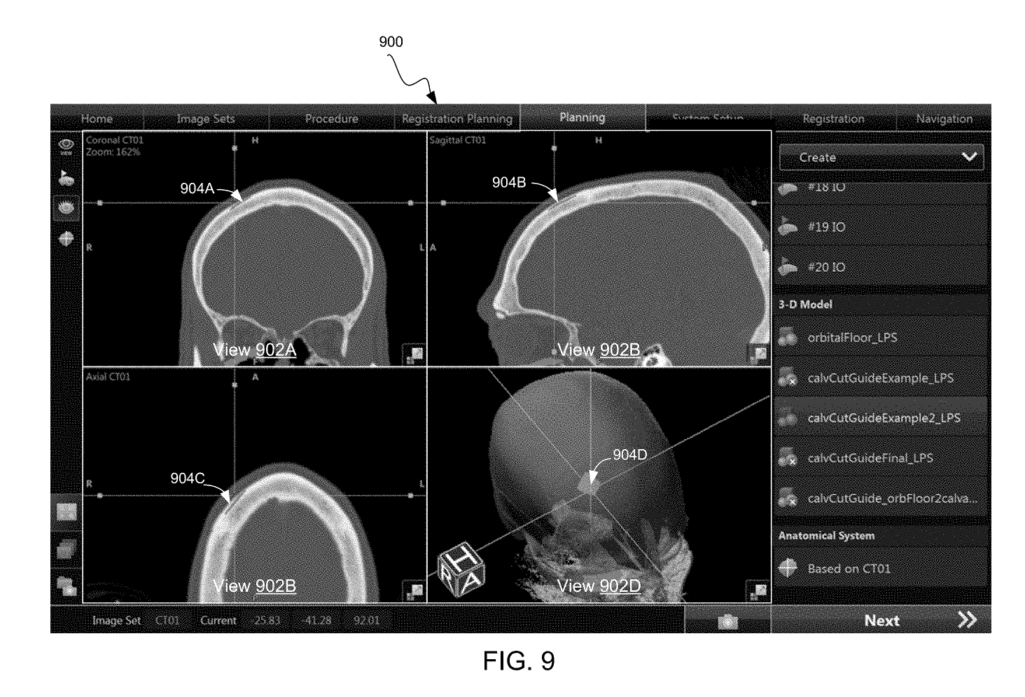

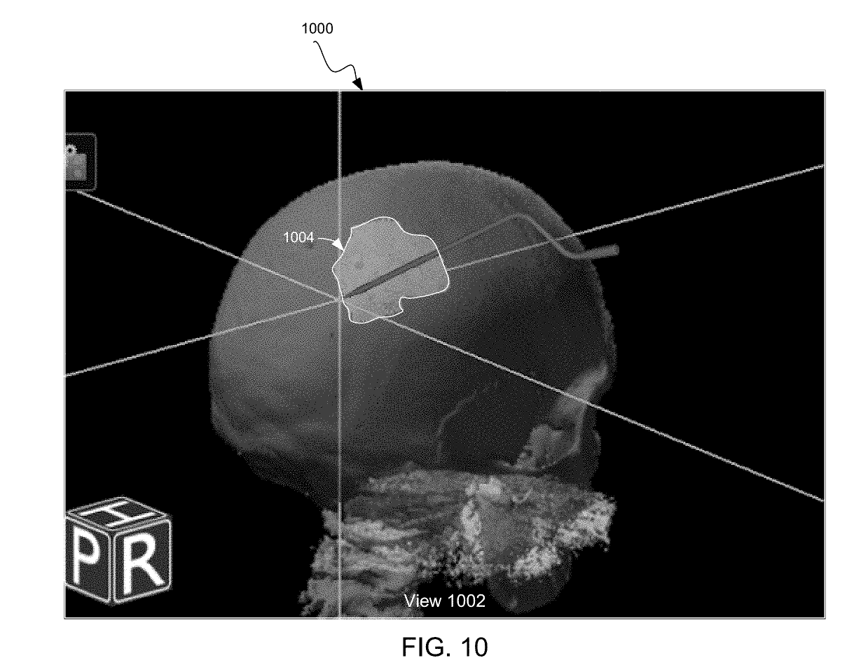

By way of example, FIGS. 9 and 10 show user interfaces 900 and 1000 of a surgical navigation system, respectively, after the stereolithographic model 802 has been uploaded. In particular, user interface 900 includes views 902A, 902B, 902C, and 902D of the patient's head showing respective perspectives 904A, 904B, 904C, and 904D of the stereolithographic model positioned within the determined donor site within the three-dimensional representation of the patient. FIG. 10 shows view 1002 of the patient's head with the stereolithographic model 1004 positioned within the determined donor site within the three-dimensional representation of the patient. Such views provide the surgeon a navigational guide for harvesting of the bone graft.

In some cases, a bone graft is modified (i.e., cut) in an attempt to improve the fit between the free flap and defect. Lines marking one or more osteotomies to modify the bone graft are overlaid on the stereolithographic model 802 so as to provide a guide for the surgeon to modify the bone graft. Such lines may be added to the virtual template before or after the bone graft is resected from the patient.

The computing system 100 may also cause fabrication of a physical model of the generated virtual template. For instance, the computing system 100 may provide the generated virtual template (e.g., a stereolithographic model) to a stereolithography machine to cause the stereolithography machine to fabricate a physical model of the virtual template using additive manufacturing. Alternatively, a numerically controlled machine tool such as a multi-axis Computerized Numerically Controlled CNC mill, fabricates a physical model of the virtual template from metal such as aluminum. A physical model may be fabricated using other techniques as well, such rapid prototyping.

Two possible advantages of subtractive (CNC machining) formation of the template are 1) CNC machining is generally faster than stereolithography in generating a model and 2) metals such as aluminum are readily subjective to the heat of common hospital sterilization equipment. Speed can be important if the virtual template is generated during surgery because the patient remains under anesthesia and with open incisions during fabrication. Sterilization is essential for surgical devices in use and aluminum or similar metals used in CNC machining are known to tolerate autoclaves or gas sterilization while use of materials used in 3D printing and stereolithography may involve additional qualification or modification. Further, CNC machining can be performed on pre-formed template blanks which have the generally correct shape for the cutting guide, but lack patient specific features such as bone shape (e.g., matched to a point cloud) and the specific cutting planes for the tissue graft. Accordingly, fabrication of a physical model using CNC machining may involve modification of a template blank to fine-tune the bone matching shape to the donor site and/or to cut slots for a bone saw which will define planes in the donor bone based on the graft site.

The surgeon may use this physical model as a template in reconstructing the defect, as the physical model can be placed in the defect to verify fit. The surgeon may also use this physical model as a guide for resecting a bone graft corresponding to the physical model (e.g., to verify that his planned osteotomies will produce a bone graft matching the physical model).

As a navigational guide for reconstructing the defect using the harvested bone graft, computing system 100 may cause the graphical display of the surgical navigation system to display the generated virtual template positioned into the defect within the three-dimensional representation. Computing system 100 may determine a geometric transformation of the generated virtual template to position the generated virtual template into the defect within the three-dimensional representation of the patient. The geometric transformation may be based on the output of the geometric alignment algorithm. Alternatively, computing system may receive input data indicating a geometric transformation of the generated virtual template display the generated virtual template concurrently with the three-dimensional representation according to the defect.

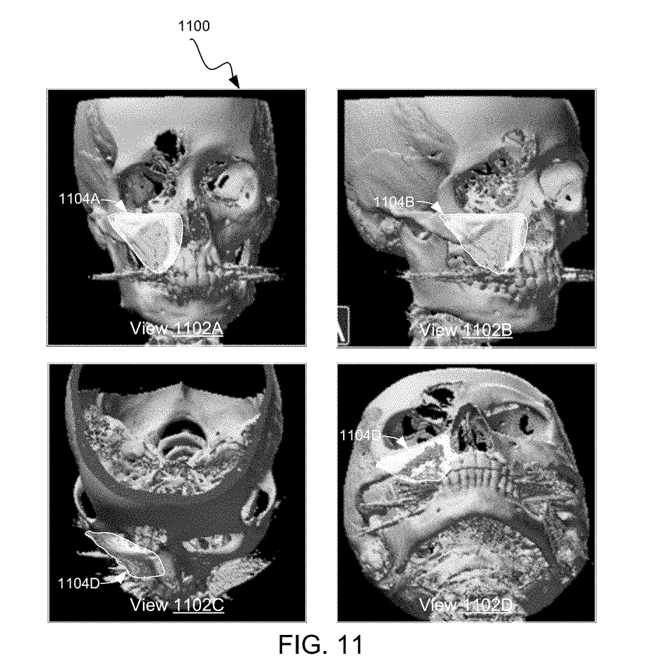

To illustrate, FIG. 11 show a user interface 1100 of a surgical navigation system. FIG. 11 shows views 1102A, 1102B, 1102C, and 1102D of the patient's head showing respective perspectives 1104A, 1104B, 1104C, and 1104D of the stereolithographic model positioned into the defect within the three-dimensional of the patient. The surgeon may such views as a navigational guide for reconstructing the defect using the harvested bone graft.

In some examples, computing system 100 may modify the three-dimensional representation of the patient undergoing the surgery to represent the defect. For instance, as noted above, the computing system may receive input data indicating one or more osteotomies made to the surgery. Computing system 100 may modify the three-dimensional representation to reflect the one or more osteotomies (e.g., by virtually removing portions of the three-dimensional representation marked by the one or more osteotomies). As such, while the imaging data is captured pre-operatively, the three-dimensional representation is modified intraoperatively to reflect the surgical procedure. Such a modification may facilitate displaying the generated virtual template positioned into the defect within the three-dimensional representation of the patient as a navigational guide for reconstructing the defect using the harvested bone graft.

### 3. EXAMPLE METHOD

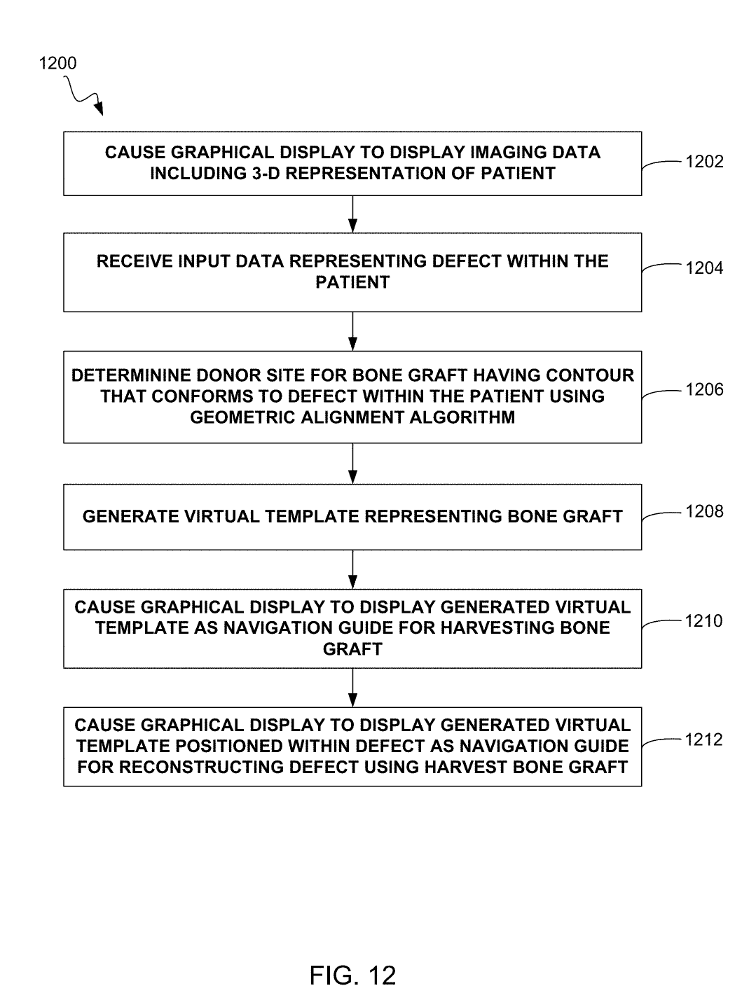

FIG. 12 shows a flowchart depicting functions that can be carried out in accordance with at least one embodiment of an example method 1200.

As shown in FIG. 12, method 1200 includes, at block 1202, causing a graphical display to display imaging data including a three-dimensional representation of a patient undergoing surgery. For instance, a computing system may cause a graphical display of a surgical navigation system to display imaging data including the three-dimensional representation of a patient undergoing surgery.

At block 1204, method 1200 involves receiving input data representing a defect within the patient. For instance, the computing system may receive input data representing one or more osteotomies made during the surgery to form a defect within the patient.

At block 1206, method 1200 involves determining a donor site for a bone graft having a contour that conforms to the defect within the patient using a geometric alignment algorithm. For instance, the computing system may determine, via one or more processors, a donor site for a bone graft having a contour that corresponds to the defect within the patient.

At block 1208, method 1200 involves generating a virtual template representing the bone graft. For example, the computing system may generate a virtual template representing the bone graft.

At block 1210, method 1200 involves causing the graphical display to display the generated virtual template as a navigational guide for harvesting the bone graft. For instance, the computing system may cause the graphical display of the surgical navigation system to display the generated virtual template representing the bone graft within the three-dimensional representation of the patient as a navigational guide for harvesting of the bone graft.

At block 1212, method 1200 involves causing the graphical display to display the generated virtual template positioned within the defect as a navigational guide for reconstructing the defect using the bone graft. For example, the computing system may cause the graphical display of the surgical navigation system to display the generated virtual template positioned into the defect within the three-dimensional representation of the patient as a navigational guide for reconstructing the defect using the harvested bone graft

In some implementations, method 1200 may be carried out entirely, or in part, by computing system 100. Other suitable computing systems may be used as well.

### 4. CONCLUSION

While various aspects and embodiments have been disclosed herein, other aspects and embodiments will be apparent to those skilled in the art. For example, with respect to the flow charts depicted in the figures and discussed herein, functions described as blocks may be executed out of order from that shown or discussed, including substantially concurrent or in reverse order, depending on the functionality involved. Further, more or fewer blocks and/or functions may be used and/or flow charts may be combined with one another, in part or in whole.

The various aspects and embodiments disclosed herein are for purposes of illustration and are not intended to be limiting, with the true scope and spirit being indicated by the following claims. Other embodiments can be utilized, and other changes can be made, without departing from the spirit or scope of the subject matter presented herein.

We claim:

1. A method comprising:
causing a graphical display of a surgical navigation system to display imaging data comprising a three-dimensional representation of a patient undergoing surgery;
receiving input data representing one or more osteotomies made during the surgery to form a defect within the patient;
determining, via one or more processors, a donor site for a bone graft having a contour that corresponds to the defect within the patient, wherein determining the donor site for the bone graft having the contour that corresponds to the defect within the patient comprises using a geometric alignment algorithm to identify, as the donor site, a portion of the bone graft that virtually aligns with the defect;
generating a virtual template representing the bone graft;
causing the graphical display of the surgical navigation system to display the generated virtual template representing the bone graft within the three-dimensional representation of the patient as a navigational guide for harvesting of the bone graft; and
causing the graphical display of the surgical navigation system to display the generated virtual template positioned into the defect within the three-dimensional representation of the patient as a navigational guide for reconstructing the defect using the harvested bone graft.

2. The method of claim 1, wherein using the geometric alignment algorithm to identify, as the donor site, the portion of the bone graft that virtually aligns with the defect comprises:
using the geometric alignment algorithm on multiple donor bones to identify, as prospective donor sites, respective portions of the multiple donor bones that virtually align with the defect; and
selecting, as the donor site, a portion of a given donor bone that virtually aligns with the defect.

3. The method of claim 1, wherein using the geometric alignment algorithm to identify, as the donor site, the portion of the bone graft that virtually aligns with the defect comprises:
generating a first point cloud representing the defect within the patient and a second point cloud representing a donor bone; and
causing the geometric alignment algorithm to identify, as the donor site, a particular portion of the donor bone represented in the second point cloud that aligns with the defect represented in the first point cloud.

4. The method of claim 3, wherein using the geometric alignment algorithm to identify, as the donor site, the particular portion of the second point cloud that aligns with the defect represented in the first point cloud comprises applying, to the geometric alignment algorithm, a weighing function that increases a weight of one or more first bone features represented in the first point cloud relative to one or more second bone features represented in the first point cloud to cause the geometric alignment algorithm to identify, as the donor site, a particular portion of the second point cloud that aligns with the one or more first bone features represented in the first point cloud.

5. The method of claim 3, wherein the geometric alignment algorithm is an iterative closest point algorithm, and wherein using the geometric alignment algorithm to identify, as the donor site, the portion of the donor bone represented in the first point cloud that aligns with the defect represented in the first point cloud comprises providing the first point cloud and second point cloud as input to the iterative closest point alignment algorithm to yield a geometric transformation that, when applied to the second point cloud, aligns the donor bone represented in the second point cloud with the defect represented in the first point cloud.

6. The method of claim 1, wherein receiving the input data representing one or more osteotomies made during the surgery to form the defect within the patient comprises receiving input data representing a set of vertices within the imaging data that represent the one or more osteotomies made during the surgery to form the defect within the patient.

7. The method of claim 1, wherein receiving the input data representing one or more osteotomies made during the surgery to form the defect within the patient comprises receiving input data representing input via a navigation pointer of the surgical navigation system to define three-dimensional coordinates on the three-dimensional representation of the patient, the three-dimensional coordinates defining the one or more osteotomies made during the surgery to form the defect within the patient.

8. The method of claim 1, further comprising:
modifying the three-dimensional representation of the patient undergoing the surgery to represent the patient after the one or more osteotomies made during the surgery, thereby representing the defect within the three-dimensional representation of the patient.

9. The method of claim 1, further comprising:
determining, via the one or more processors, a geometric transformation of the generated virtual template to position the generated virtual template into the defect within the three-dimensional representation of the patient.

10. The method of claim 1, wherein the donor site is determined after the one or more osteotomies are made during the surgery.

11. The method of claim 1, further comprising:
causing fabrication of a physical model of the generated virtual template, wherein fabricating the physical model comprises at least one of (i) causing a three-dimensional printer to fabricate the physical model of the generated virtual template or (ii) causing a numerically controlled machine tool to fabricate the physical model of the generated virtual template.

12. A system comprising:
one or more processors;
a communications interface to a surgical navigation system; and
a tangible non-transitory computer-readable medium having computer-executable instructions stored thereon that are executable by one or more processors of a computing device to cause the computing device to perform functions comprising:
causing a graphical display of the surgical navigation system to display imaging data comprising a three-dimensional representation of a patient undergoing a surgery;
receiving input data representing one or more osteotomies made during the surgery to form a defect within the patient;
determining, via one or more processors, a donor site for a bone graft having a contour that corresponds to the defect within the patient, wherein determining the donor site for the bone graft having the contour that corresponds to the defect within the patient comprises using a geometric alignment algorithm to identify, as the donor site, a portion of the bone graft that virtually aligns with the defect;
generating a virtual template representing the bone graft;
causing the graphical display of the surgical navigation system to display the generated virtual template representing the bone graft within the three-dimensional representation of the patient as a navigational guide for harvesting of the bone graft; and
causing the graphical display of the surgical navigation system to display the generated virtual template positioned into the defect within the three-dimensional representation of the patient as a navigational guide for reconstructing the defect using the harvested bone graft.

13. The system of claim 12, wherein using the geometric alignment algorithm to identify, as the donor site, the portion of the bone graft that virtually aligns with the defect comprises:
using the geometric alignment algorithm to identify, as prospective donor sites, respective portions of multiple donor bone that virtually align with the defect; and
selecting, as the donor site, a portion of a given donor bone that virtually aligns with the defect.

14. The system of claim 12, wherein using the geometric alignment algorithm to identify, as the donor site, the portion of the bone graft that virtually aligns with the defect comprises:
generating a first point cloud representing the defect within the patient and a second point cloud representing a donor bone; and
causing a geometric alignment algorithm to identify, as the donor site, a particular portion of the donor bone represented in the second point cloud that aligns with the defect represented in the first point cloud.

15. The system of claim 14, wherein using the geometric alignment algorithm to identify, as the donor site, the particular portion of the second point cloud that aligns with the defect represented in the first point cloud comprises applying, to the geometric alignment algorithm, a weighing function that increases a weight of one or more first bone features represented in the first point cloud relative to one or more second bone features represented in the first point cloud to cause the geometric alignment algorithm to identify, as the donor site, a particular portion of the second point cloud that aligns with the one or more first bone features represented in the first point cloud.

16. The system of claim 14, wherein the geometric alignment algorithm is an iterative closest point algorithm, and wherein using the geometric alignment algorithm to identify, as the donor site, the portion of the donor bone represented in the first point cloud that aligns with the defect represented in the first point cloud comprises providing the first point cloud and second point cloud as input to the geometric alignment algorithm to yield a geometric transformation that, when applied to the second point cloud, aligns the donor bone represented in the second point cloud with the defect represented in the first point cloud.

17. The system of claim 12, wherein receiving the input data representing one or more osteotomies made during the surgery to form the defect within the patient comprises receiving input data representing a set of vertices within the imaging data that represent the one or more osteotomies made during the surgery to form the defect within the patient.

18. The system of claim 12, wherein receiving the input data representing one or more osteotomies made during the surgery to form the defect within the patient comprises receiving input data representing input via a navigation pointer of the surgical navigation system to define three-dimensional coordinates on the three-dimensional representation of the patient, the three-dimensional coordinates defining the one or more osteotomies made during the surgery to form the defect within the patient.

19. The system of claim 12, the functions further comprising:
causing fabrication of a physical model of the generated virtual template, wherein fabricating the physical model comprises at least one of (i) causing a three-dimensional printer to fabricate the physical model of the generated virtual template or (ii) causing a numerically controlled machine tool to fabricate the physical model of the generated virtual template.

20. A tangible non-transitory computer-readable medium having computer-executable instructions stored thereon that are executable by one or more processors of a computing system to cause the computing system to perform a method comprising:
causing a graphical display of a surgical navigation system to display imaging data comprising a three-dimensional representation of a patient undergoing surgery;
receiving input data representing one or more osteotomies made during the surgery to form a defect within the patient;
determining, via one or more processors, a donor site for a bone graft having a contour that corresponds to the defect within the patient, wherein determining the donor site for the bone graft having the contour that corresponds to the defect within the patient comprises using a geometric alignment algorithm to identify, as the donor site, a portion of the bone graft that virtually aligns with the defect;
generating a virtual template representing the bone graft;
causing the graphical display of the surgical navigation system to display the generated virtual template representing the bone graft within the three-dimensional representation of the patient as a navigational guide for harvesting of the bone graft; and
causing the graphical display of the surgical navigation system to display the generated virtual template positioned into the defect within the three-dimensional representation of the patient as a navigational guide for reconstructing the defect using the harvested bone graft.
